(19) United States
(12) Patent Application Publication
(10) Pub. No.: US 2009/0270439 A1
(43) Pub. Date: Oct. 29, 2009

(54) Prophylactic/therapeutic agent for alzheimer's disease

(76) Inventor: Yasumasa Ohyagi, Fukuoka-shi (JP)

Correspondence Address: WENDEROTH, LIND & PONACK, L.L.P., 1030 15th Street, N.W.,, Suite 400 East, Washington, DC, 20005-1503, US

(21) Appl. No.: 12/232,372

(22) Filed: Sep. 16, 2008

Related U.S. Application Data
(63) Continuation-in-part of application No. PCT/JP2007/055354, filed on Mar. 16, 2007.

(30) Foreign Application Priority Data
Mar. 16, 2006 (JP) 2006-073054

(51) Int. Cl. A61K 31/473; A61P 25/28

(52) U.S. Cl. 514/284

(57) ABSTRACT

A medicament effective for inhibiting onset of Alzheimer disease via intracellular aggregation mechanism is provided. A medicament for prophylaxis and treatment of Alzheimer disease comprising as an active ingredient apomorphine hydrochloride wherein cell death dependent upon intracellularly accumulated amyloid β proteins and/or p53 is inhibited; and/or wherein a level of intracellularly accumulated amyloid β proteins and/or phosphorylated tau proteins is lowered to improve energy production, protein metabolism and synaptic function of neurons so as to ameliorate the recognition capacity such as defects of memory. A medicament for prophylaxis and treatment of Alzheimer disease according to the present invention may allow for eradicative treatment of Alzheimer disease due to its inhibition of intracellular coagulation and accumulation of amyloid β proteins and intracellular activation of proteasome, and even prior to occurrence of massive neuronal death, may improve the function per se of neurons by reducing intracellularly accumulated amyloid β proteins to thereby contribute amelioration of the recognition function of Alzheimer disease patients.

### TECHNICAL FIELD

The present invention relates to a medicament for prophylaxis and treatment of Alzheimer disease comprising as an active ingredient apomorphine hydrochloride. More specifically, the present invention relates to a medicament for prophylaxis and treatment of Alzheimer disease comprising as an active ingredient apomorphine hydrochloride wherein the medicament acts through inhibition of intracellular accumulation of amyloid β and/or p53 dependent cell death.

### BACKGROUND ART

Alzheimer disease is in particular a serious issue in Japan where dwindling birthrate is remarkable and aging society has progressed, as its number of patients keeps on increasing (there are 1.6 to 1.8 million patients as of 2005 in Japan). Although research of Alzheimer disease has extensively been done for addressing this issue, an eradicative medicine has not been developed that allows for determent of neuronal death, the key to the development of the disease.

A hypothesis of amyloid cascade has been proposed as a mechanism of onset of Alzheimer disease. According to this hypothesis, an amyloid β protein precursor (hereinafter also referred to as “APP”) is cleaved by β- and γ-secretases to produce an amyloid β protein (hereinafter also referred to as “Aβ”) which aggregates and accumulates to thereby cause destruction of cerebral neurons and fall-off of the cerebral nerve. An amyloid β protein precursor, a parent glycoprotein of an amyloid β protein that is a major cause of Alzheimer disease, remains to be elucidated for its function. On the other hand, an amyloid β protein is a major component of “senile plaque” typically found in Alzheimer disease patients with a molecular weight of about 4 kDa, among which three types of Aβ40, Aβ42 and Aβ43 are known based on the number of amino acid residues. It is known that an amyloid β protein as a major cause of Alzheimer disease may have a low cytotoxicity in its monomer form but exert a potent toxicity in its oligomer form upon aggregation. Aggregation of an amyloid β protein causes destruction of cerebral neurons and fall-off of the cerebral nerve to thereby form amyloid plaques and neurofibrillary changes, which trigger the cause of cell death of cerebral neurons and fall-off of neurons such as acetylcholinergic neurons, ultimately resulting in onset of Alzheimer disease.

A medicament approved to be efficacious to symptoms characteristic to Alzheimer disease such as recognition disturbance and defects of memory includes chiefly one increasing an acetylcholine level in the brain, i.e. a choline esterase inhibitor, based on the notion that intracerebral disturbance of acetylcholinergic nerve in patients suffering from Alzheimer disease is the cause of the disease. There is only one such a medicament in Japan, “ARICEPT” (Donepezil HCI; Eisai Co., Ltd.), which is a choline esterase inhibitor. The sales of ARICEPT in Japan at the first quarter of 2005 are 35.1 billion yen, 23% higher than the previous year, and 162.9 billion yen worldwide, 15% higher than the previous year. However, ARICEPT merely restrictedly suppresses the aggravation of symptoms of Alzheimer disease but is never an eradicative medicine of the disease. It is estimated that only 47% of Alzheimer disease patients in Japan actually undergo therapeutic treatment and thus a market of a medicament for treating dementia is expected to keep growing from now on to accelerate the research and development of the drugs.

With the knowledge that neurodegeneration progresses by neurotoxicity of an extracellular amyloid β protein, especially Aβ42, therapeutic strategy many researchers and enterprises currently envisage includes, for preventing the neurodegeneration, an inhibitor of β- and γ-secretases that produce an amyloid β protein, an activator of neprilysin (RIKEN) that degrades an extracellular amyloid β protein, an inhibitor of polymerization of an amyloid β protein, and in particular vaccination. For vaccination, an amyloid β protein is administered to patients as a vaccine so as to produce an antibody thereto wherein said antibody would remove senile plaques and inhibit aggregation and deposition of secreted amyloid β proteins to thereby prevent fall-off of neurons. In either therapeutic medicament, however, severe adverse side effects such as encephalitis are observed and neurodegeneration per se is not actually inhibited and thus eradication of Alzheimer disease would not be possible.

Recently, toxicity of amyloid β proteins (Aβ42) accumulated within neurons has been focused and, as a mechanism, its action at endoplasmic reticulum, mitochondria and synapse has been proposed in several treatises (Non-patent references 1, 2, 3 and 4). However, the action at nucleus or a mechanism directly leading to apoptosis has not yet been reported.

The accumulation of amyloid β proteins (Aβ42) within neurons is induced by excessive oxidative stress such as hydrogen peroxide associated with aging of the brain and, although neurons do not die at this stage, risk leading to apoptosis is quite increased. When amyloid β proteins (Aβ42) accumulated within neurons are transferred to nucleus, cell death is accelerated through increased expression of p53.

Recently, Aβ-related Death Inducing Protein (AB-DIP) has been identified as a molecular chaperone that transfers Aβ from cytoplasm to nucleus (Non-patent reference 5). Functional acceleration of this molecule would induce apoptosis of cells which is known to be Aβ dependent. Therefore, not only damages in mitochondria, endoplasmic reticulum and synapse but also a pathway of Aβ transfer to nucleus as directly leading to p53 dependent apoptosis is thought to be most important.

Viewing that acceleration of intracellular Aβ42/p53 dependent apoptosis may deeply be involved in fall-off of neurons observed in as high as 90% or more of the brain affected with Alzheimer disease, blockage of this pathway would extremely be important. As a strategy, it is considered to be rational to accelerate degradation of Aβ accumulated in cytoplasm and of p53 by activation of proteasome function. The reason for this includes that: 1) Aβ accumulated in cytoplasm may possibly damage mitochondria and synapse even not through apoptosis; 2) functional decline of proteasome in neurons due to aging or Alzheimer disease is reported (Non-patent reference 6); 3) it will be convenient to target proteasome since proteasome degrades both Aβ42 and p53; and 4) accumulation of abnormal proteins in neurons has been observed not only in Alzheimer disease but also in e.g. Parkinson disease or spinocerebellar degeneration and thus suppression of neurodegenerative mechanism in general may be expected.

Proteasome is a gigantic multi-component complex consisted of about 50 subunits in total having a molecular weight of about 2 million and is said to be the biggest and the most complex enzyme in the history of biochemistry. It localizes in nucleus and cytoplasm and selectively degrades intracellular proteins. Its main targets are many proteins involved in cell cycle, growth and apoptosis. Most of proteins of short life are degraded through ubiquitin/proteasome pathway. This ubiquitin/proteasome pathway is deeply involved in various life phenomena such as metabolism, cell cycle, apoptosis, positive/negative signaling, quality control of proteins, stress and immunological response and draws attention as being a new control system of living reactions different from the control by biosynthesis of proteins as recognized so far. It is well foreseeable that breakdown of this control system will lead to the cause of various pathological conditions and therefore research of a medicament that may positively and/or negatively control this control system would contribute to development of therapy efficacious for a variety of intractable diseases currently difficult to deal with.

Since 1869 when apomorphine hydrochloride was initially used as an emetic, it has been used as a sedative for patients suffering from schizophrenia and also as a behavior-improving agent for patients suffering from alcoholic or narcotic intoxication during the first half of the 21st century. In Japan, it has been described in Japan Pharmacopoeia from the 1st edition (issued in 1886) up to the 7th revision (issued in 1966; Part 2) and also in National Formulary. It has been clinically used as an emetic with a high dosage (normal dose: 5 mg, subcutaneous; maximum dose: 20 mg, subcutaneous) or as an expectorant with a low dosage (0.5-1 mg/dose). In 1967, apomorphine hydrochloride was found to be efficacious as a dopamine agonist and begun to be clinically used as an anti-Parkinson disease agent. In Europe, it is currently used as an anti-Parkinson disease agent (subcutaneous; 1.5-10 mg/dose, 2-8 doses/day).

Relevancy between apomorphine hydrochloride and Alzheimer disease is not known up till the present. It is reported that a certain apomorphine analogue accelerates oligomerization of amyloid β proteins and inhibits fibrillization thereof (Non-patent reference 7). However, this report merely concerns the action and mechanism in the extracellular amyloid cascade hypothesis. There is also a report that a glycoside and a orthoester glycoside derivative of apomorphine and analogue is used for treatment and amelioration of disorders including Alzheimer disease and amnesia and/or dementia (Non-patent references 1 and 2). However, this report focuses on the treatment of functional disorder of erection but fails to teach any of the action and mechanism against Alzheimer disease or pharmacological effects.

Patent reference 1: Japanese Patent Publication No. 2005-526790
Patent reference 2: WO 03/080074
Non-patent reference 1: Lustbader, J. W. et al., Science Vol. 304, No. 5669, p. 448-452, 2004
Non-patent reference 2: Yan, S. D. & Stern, D. M., Int. J. Exp. Pathol., Vol. 86, No. 3, p. 161-171, 2005
Non-patent reference 3: Takahashi, R. H. et al., Am. J. Pathol., Vol. 161, No. 5, p. 1869-1879, 2002
   Non-patent reference 4: Borghi, R. et al., J. Alzheimer Dis., Vol. 4, No. 1, p. 31-37, 2002
   Non-patent reference 5: Lakshmana, M. K. et al., FASEB J., Vol. 19, No. 10, p. 1362-1364, 2005
   Non-patent reference 6: Lopez Salon, M. et al., J. Neurosci. Res., Vol. 62, No. 2, p. 302-310, 2000
   Non-patent reference 7: The Journal of Biological Chemistry, Vol. 277, No. 45, p. 42881-42890, 2002

### DISCLOSURE OF THE INVENTION

### Technical Problem to be Solved by the Invention

An object of the present invention is to provide a novel medicament for prophylaxis and treatment of Alzheimer disease, the disease no possibility for eradication has been presented to, based on a novel action and mechanism.

### Means for Solving the Problems

For solving the problems mentioned above, the present inventor has stained and carefully observed the brain of patients suffering from Alzheimer disease with an anti-Aβ antibody and found that, in addition to classical senile plaques, Aβ was accumulated within neurons and that there were two patterns of Aβ42 accumulation within neurons, i.e. cytoplasmic and nuclear accumulations. The present inventor has further found that cytoplasmic accumulation of Aβ occurs at an early stage and a part thereof is transferred to nucleus to thereby render the neuron die out soon, resulting in formation of senile plaques. Based on this finding, the present inventor has firstly found that, apart from extracellular aggregation and accumulation of amyloid β proteins that has been considered to be an initiation mechanism of Alzheimer disease, increase in a level of amyloid β proteins and acceleration of the p53 cascade in cells, especially in nucleus, would play an important role in the onset of the disease and has earnestly studied for searching for a medicament efficacious for inhibiting the onset of the disease by targeting the intracellular accumulation mechanism. As a result, the present inventor has found that, surprisingly, apomorphine hydrochloride affected the intracellular accumulation mechanism to effectively inhibit neuron death in Alzheimer disease to thereby complete the present invention. Specifically, apomorphine hydrochloride, as an active ingredient of a medicament for prophylaxis and treatment of Alzheimer disease of the present invention, efficaciously prevent and treat Alzheimer disease by acting to an intracellular proteasome for activation of its function to thereby accelerate degradation of both intracellular Aβ and p53 proteins so as to inhibit apoptosis of neurons as well as to inhibit damage in mitochondria/synapse.

Apomorphine hydrochloride was tested by subcutaneous injection to 3×Tg mouse, one of model mice of Alzheimer disease. Apomorphine hydrochloride was administered weekly for a month to 3×Tg mouse of 6 months old with defects of short-term memory characteristic to Alzheimer disease and its recognition capacity of space memory was quantitatively analyzed with Morris water maze test before and after the treatment. After completion of the Morris test with administration, a slice of the brain was prepared and an inhibitory effect to accumulation of intracellular amyloid β proteins and phosphorylated tau proteins was pathologically evaluated by immunostaining. As a result, apparent improvement was observed in both the memory test and the immunostaining to demonstrate that apomorphine hydrochloride was efficacious for recovery of recognition capacity such as memory in patients suffering from Alzheimer disease. Also, it was found that injection of apomorphine hydrochloride to 3×Tg mouse at 3 months old, wherein defects of recognition capacity has not yet been presented, inhibited the onset of the defects when the mouse grew 6 months old, suggesting the prophylactic effect of apomorphine hydrochloride to Alzheimer disease.

The present invention relates to a medicament for prophylaxis and treatment of Alzheimer disease comprising as an active ingredient apomorphine hydrochloride. More specifically, the present invention relates to a medicament for prophylaxis and treatment of Alzheimer disease comprising as an active ingredient apomorphine hydrochloride wherein (1) cell death dependent upon intracellularly accumulated amyloid β proteins and/or p53 is inhibited; and/or (2) a level of intracellularly accumulated amyloid β proteins and/or phosphorylated tau proteins is lowered to improve energy production, protein metabolism and synaptic function of neurons so as to ameliorate the recognition capacity such as defects of memory.

(More Efficacious Effects than Prior Art)

A medicament for prophylaxis and treatment of Alzheimer disease comprising as an active ingredient apomorphine hydrochloride according to the present invention may reduce amyloid β proteins and p53 proteins accumulated within neurons, rather than inhibition of coagulation and accumulation of extracellularly accumulated amyloid β proteins as conventionally viewed. Since the intracellular coagulation and accumulation of amyloid β proteins are thought to be the direct cause of massive neuronal death in Alzheimer disease, by enhancing degradation of such amyloid β proteins, the medicament for prophylaxis and treatment of Alzheimer disease of the present invention may exert prominent effects to inhibit neuronal death. Thus, as compared to a coagulation inhibitor of extracellular amyloid β proteins, the medicament according to the present invention allows for more eradicative prophylaxis and treatment of Alzheimer disease by inhibiting massive neuronal death.

On the other hand, it is known that the amyloid β proteins accumulated within neurons, other than induction of neuronal death via p53 dependent apoptosis, may also be involved in damage to energy production in mitochondria (Non-patent reference 1), damage to synaptic function (Non-patent reference 3), and damage to proteasome function (Almeida CG et al., J. Neurosci. 26: 4277-4288 (2006)). In 3×Tg mice, after administration of apomorphine hydrochloride, fall-off of massive neurons was not observed even at the stage of 6-month when amyloid β proteins are accumulated within neurons of the hippocampus and the cerebral cortex. Thus, as a mechanism of improvement in memory power as a consequence of administration of apomorphine hydrochloride, it was suggested that the function of various neurons such as the hippocampus was recovered through decrease in intracellular amyloid β proteins. This also suggests that intracellular accumulation of amyloid β proteins is a preliminary stage of p53 dependent apoptosis and that activation of a chaperon protein called AB-DIP, which transfers amyloid β proteins from cytoplasm to nuclear, would be necessary for inducing massive apoptosis (Non-patent reference 5; Ohyagi Y. et al., Mini. Rev. Med. Chem. 6: 1075-1080 (2006)). Accordingly, even prior to occurrence of massive neuronal death, apomorphine hydrochloride may improve the function per se of neurons by reducing intracellularly accumulated amyloid β proteins to thereby contribute amelioration of recognition function of Alzheimer disease patients.

### BRIEF DESCRIPTION OF DRAWINGS

FIG. 1 is a graph showing by quantitative evaluation of immune reaction the results of the test where the effect of apomorphine hydrochloride (APO) on degradation of intracellularly accumulated Aβ40 was investigated in a model of intracellularly accumulated amyloid β proteins.

FIG. 2 shows that intracellular Aβ40 is degraded with lapse of time by addition of apomorphine hydrochloride (APO) in cell culture.

FIG. 3 shows the cell death inhibitory activity of apomorphine hydrochloride (APO) to cell death induced by hydrogen peroxide.

FIG. 4 shows the cell death inhibitory activity of apomorphine hydrochloride (APO) to cell death induced by hydrogen peroxide. Panel A indicates a change in p53 level after addition of apomorphine hydrochloride. Panel B indicates a change in a survival rate of cells after addition of apomorphine hydrochloride.

FIG. 5 is a graph showing by quantitative evaluation of immune reaction the results of the test where the effect of apomorphine hydrochloride (APO) on degradation of intracellularly accumulated Aβ42 was investigated in a model of intracellularly accumulated amyloid β proteins.

FIG. 6 shows that apomorphine hydrochloride (APO) alone enhanced the proteasome activity and that the decreased proteasome activity at addition of oxidative stress was recovered by adding apomorphine hydrochloride (APO) in SH-SY5Y cells.

FIG. 7 shows that the apoptosis inhibitory activity of apomorphine hydrochloride (APO) observed for oxidative stress in SH-SY5Y cells was also seen in primary neurons from mouse fetus, i.e. native neurons rather than cancerous cells.

FIG. 8 shows immunostaining of primary neurons from mouse fetus with an antibody to beta-tubulin, a cytoskeletal protein of the axon.

FIG. 9 illustrates principles of Morris water maze test. In an arbitrary position of a round-shaped pool with a diameter of about 1 m is set a transparent goal stand (left panel). The goal stand is submerged about 1 cm under the surface of the water and cannot be seen from outside the water. A mouse is let to start at an arbitrary position and will seek for the goal stand. After training four times a day for several consecutive days, the mouse shall learn the place of the goal stand and reach it via the shortest course (right panel).

FIG. 10 shows time (s; second) needed for reaching the goal stand in 3-day swimming training in heterozygous 3×Tg mice (n=4 in each group) where the results are shown in comparison before (before) and after (after) injection for a month. After injection for a month, a tendency of improvement in the capacity of memory and learning was observed in mice receiving 5 mg/kg of apomorphine hydrochloride (APO).

FIG. 11 shows evaluation of the memory of the goal stand position at 24 hours after the swimming training in heterozygous 3×Tg mice (n=4 in each group) (48-hour probe test). In groups of apomorphine hydrochloride (APO) injection, shortening of time lag in reaching the goal stand, increase in frequency of crossing the goal stand, and increase in residential time (%) within ¼ block of the goal stand were significantly observed. In contrast, in APO-0 group, there was no change between before and after injection or even aggravation of the memory retention function was observed.

FIG. 12 shows a 48-hour probe test of 3×Tg cases (2 mice) in which apomorphine hydrochloride (APO) injection was efficacious. Figures of indexes (right tables) and loci of swimming (drawings) are shown for each case. After injection, the memory retention of the goal stand position was improved.

FIG. 13 shows immunostaining with an anti-amyloid β antibody (4G8). As compared to control mice (received apomorphine hydrochloride (APO)-0 mg/kg, saline alone), intraneuronal accumulation of amyloid β proteins was inhibited both in the hippocampus and the cerebral cortex in mice injected with apomorphine hydrochloride (APO). Upper panel: hippocampus/cerebral cortex with small magnification; Lower panel: CA1 region of hippocampus with large magnification.

FIG. 14 shows immunostaining with an anti-phosphorylated tau antibody (AT8). As compared to control mice (received apomorphine hydrochloride (APO)-0 mg/kg, saline alone), intraneuronal accumulation of phosphorylated tau proteins was inhibited both in the hippocampus and the cerebral cortex in mice injected with apomorphine hydrochloride (APO). Upper panel: hippocampus/cerebral cortex with small magnification; Lower panel: CA1 region of hippocampus with large magnification.

FIG. 15 shows time lag in reaching the goal stand in Morris water maze test where weekly injection was continued in homozygous 3×Tg mice of 3 months old up till 6 months old. 3×Tg mice injected with 5 mg/kg of apomorphine hydrochloride (APO) had the capacity of memory and learning comparable to Non-Tg mice whereas 3×Tg mice with no APO exhibited serious defect in the capacity of learning and required 7 days for acquisition of memory.

### BEST MODE FOR CARRYING OUT THE INVENTION

Apomorphine hydrochloride as used herein as an active ingredient in a medicament for prophylaxis and treatment of Alzheimer disease of the present invention may be commercially available one (Uprima; Takeda/Abbott).

A dose of a medicament for prophylaxis and treatment of Alzheimer disease of the present invention may be a clinically effective amount that may vary depending upon severity, sex, age, body weight etc. of patients and be determined by discretion of a physician. It is usually in a range of about 0.1 mg to about 2 mg, more preferably about 0.3 mg to about 1.5 mg, and most preferably about 0.5 mg to about 1 mg.

A route of administration of a medicament for prophylaxis and treatment of Alzheimer disease of the present invention may be those commonly used without specific limitation and includes, for instance, oral administration, intraperitoneal infusion, intratracheal infusion, intrabronchial infusion, and direct intrabronchial drip, subcutaneous infusion, transdermal delivery, intraarterial infusion, intravenous infusion, intranasal administration, and the like. Among these, however, intranasal administration is preferable in favor of direct transfer of the medicament into the brain as passing through those portions of blood-brain barrier (BBB) that are comparatively thinner.

A medicament for prophylaxis and treatment of Alzheimer disease of the present invention may be prepared by the methods known in the art as appropriately met for respective routes of administration and dosage forms. By way of example, the medicament for prophylaxis and treatment to be administered orally may be in the form of capsules, solutions, and the like. Thus, the medicament for prophylaxis and treatment according to the present invention may comprise a pharmaceutically acceptable carrier, diluent, preservative, etc. as appropriately met for respective dosage forms.

The present invention is explained in more detail by means of the following Examples but should not be construed to be limited thereto.

### Example 1

### Effect of Apomorphine Hydrochloride on Degradation of Intracellularly Accumulated Aβ40

### (1) Establishment of a Model of Intracellularly Accumulated Amyloid β Proteins

In order to investigate a compound that inhibits intracellular aggregation and accumulation of amyloid β proteins based on activation of intracellular proteasome, a system was constructed for assaying an activity to degrade intracellularly accumulated Aβ. Neuroblastoma cells (SH-SY5Y; ATCC) were used as culture cells and Aβ40 was artificially accumulated within the cells. Here the reason why Aβ40 was used rather than Aβ42 was that Aβ40 is more soluble in water and hence more easily handled than Aβ42. If the activity to degrade Aβ40 was high, the activity to degrade Aβ42 was analogously thought to be high. For introducing amyloid β proteins within cells, hypertonicity influx method was used. Briefly, using Influx™ Pinocytic Cell-loading Reagent (Molecular Probe), SH-SY5Y cells (1×10⁶ cells) were exposed to Aβ40 (about 200 μg; ANASPEC) with hypertonicity for 10 minutes/hypotonicity for 2 minutes so that Aβ40 peptides may be incorporated into cells via pinocytosis and spread pervasively in cytoplasm. After accumulation of Aβ40 within cells, degradation of Aβ with lapse of time was observed. MG132 (Wako Pure Chemical Industries, Ltd.) dissolved in DMSO was used as an inhibitor that artificially inhibits the protease activity and apomorphine hydrochloride (Sigma-Aldrich; hereinafter also referred to as “APO”) was dissolved in water. These reagents were added to culture two hours before Aβ40 accumulation to investigate their activity.

### (2) Quantitative Evaluation

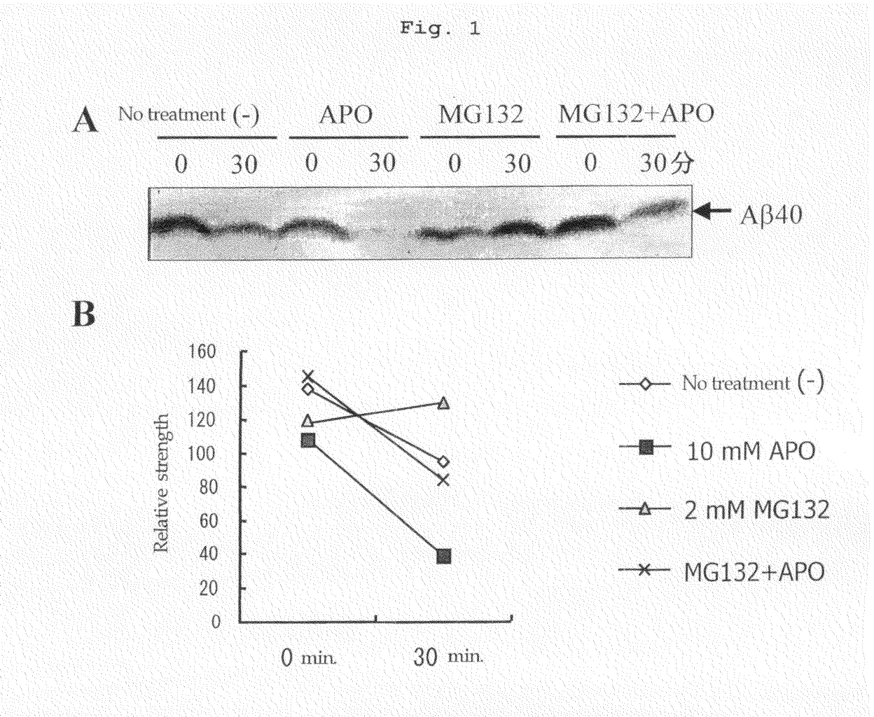

Zero minute and thirty minutes after Aβ40 treatment, cells were collected and subjected to immunoblot. Immunoblot was performed as commonly known in the art wherein soluble proteins were extracted from cells with 2% SDS, run on Tris/Tricine gel SDS-PAGE, transblotted to PVDF membrane (Semi-dry method) and detected with an anti-Aβ antibody (4G8, Signet Pathology Systems). Quantitative evaluation was done by quantitatively evaluating the immune reaction of amyloid β proteins. Quantitative evaluation was performed by taking in the photos with Photoshop and measuring density of bands with NIH Image. As a result, it was found that degradation after 30 minutes was enhanced by adding APO and that, although degradation was inhibited by adding MG132, the degradation was recovered by adding APO (FIGS. 1A, 1B). Thus, it was considered that intracellularly accumulated Aβ40 were degraded by proteasome and that APO may enhance the degrading process.

(3) Change in Intracellularly Accumulated Aβ40 with Lapse of Time in Cell Culture

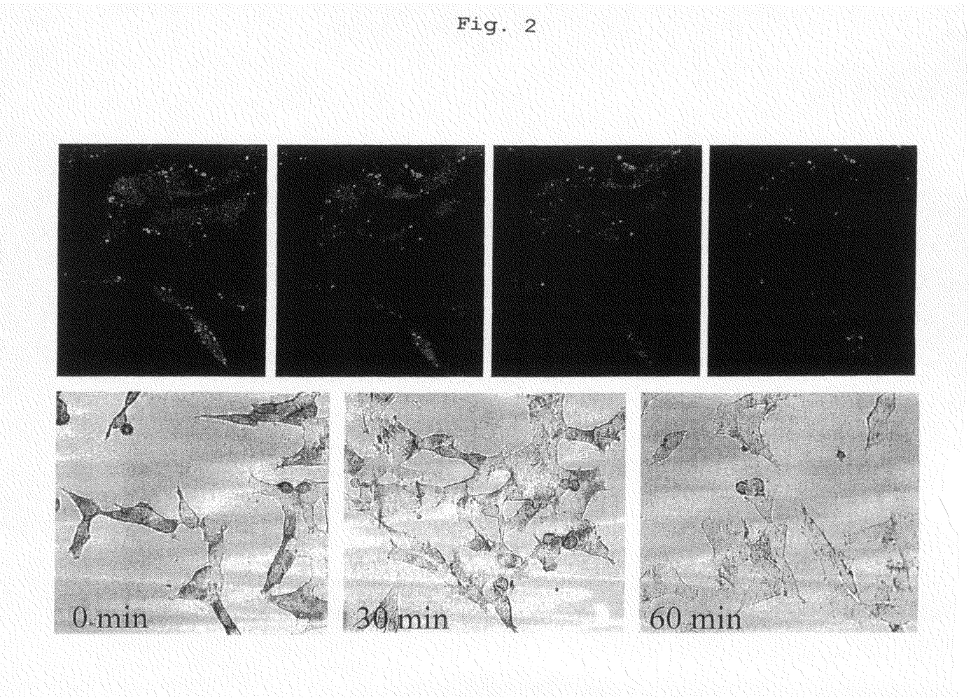

Aβ40 was incorporated into cells via pinocytosis as in (1) above. Before two hours, 10 μM APO was added to culture and then the cells were subjected to immunoblot as in (2) above and change in the level of intracellular Aβ40 was observed with lapse of time. The results are shown in FIG. 2 in which upper panels indicate Aβ40 fluorescently labeled with green fluorescence (Alexa-Fluor488) using AlexaFluor™ Protein Labeling Kit (Molecular Probe) which was observed with a confocal laser scanning microscopy (Olympus Corporation). As is clear from FIG. 2, it was observed that Aβ40 within cytoplasm was degraded with lapse of time by addition of APO.

### Example 2

### Protective Effect of APO to Apoptosis Induced by Hydrogen Peroxide

### (1) Enhancement of Cell Survival by Addition of APO

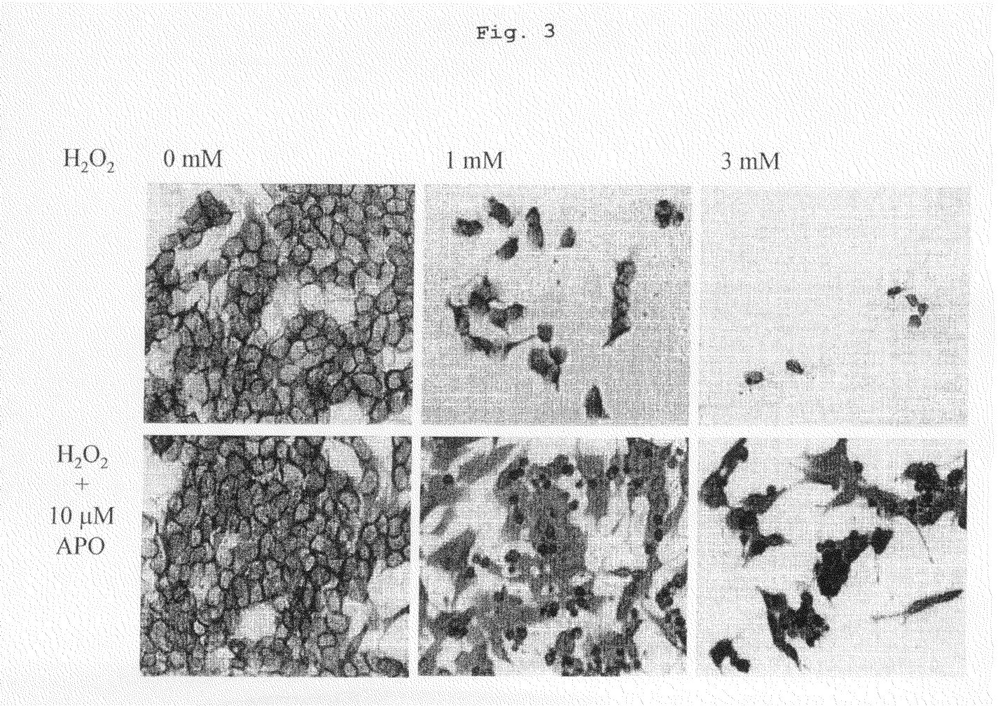

A cell death inhibitory activity of apomorphine hydrochloride to cell death induced by hydrogen peroxide was studied. Hydrogen peroxide treatment was performed by adding an appropriate amount (0 mM, 1 mM, and 3 mM) of hydrogen peroxide to normal culture medium for SH-SY5Y cells supplemented with 10% serum and by investigating cell survival after 24 hours. The cells were fixed with methanol/acetone (50%/50%) for 10 minutes so that the conditions of surviving cells could easily be observed and then subjected to immunostaining with an anti-APP C-terminal antibody (Sigma-Aldrich) using Universal Kit (DAKO). As a result, cell death was induced by hydrogen peroxide (FIG. 3, upper panels). Then, the cells were treated with 10 μM APO together with 0 mM, 1 mM, and 3 mM of hydrogen peroxide. As a result, evident enhancement of cell survival by addition of APO was observed from the number of surviving cells and the morphological features when treated with APO (FIG. 3, lower panels).

### (3) Change in p53 Level and Rate of Cell Survival by Addition of APO

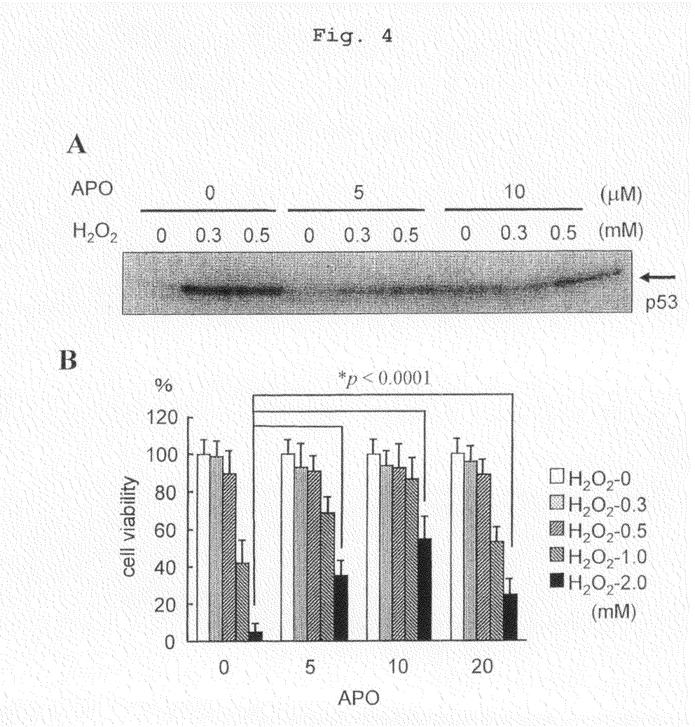

Further, in order to study p53 level and a rate of cell survival in cell damage induced by hydrogen peroxide, p53 Western blot analysis and survival rate assay using Cell Titer-Blue Assay Kit (Promega) were performed. Hydrogen peroxide treatment was done by adding an appropriate amount (0 mM, 1 mM, and 3 mM) of hydrogen peroxide to normal culture medium for SH-SY5Y cells supplemented with 10% serum as described above. After 24 hours, the increased p53 level by addition of hydrogen peroxide was inhibited by addition of APO (FIG. 4A). A rate of cell survival was also recovered by addition of APO. Thus, it was proved that APO exhibited cell death inhibitory activity to cell death induced by hydrogen peroxide. In particular, a rate of cell survival was extremely improved by addition of 10 μM APO (FIG. 4B; p<0.0001).

### Example 3

### Effect of Apomorphine Hydrochloride on Degradation of Intracellularly Accumulated Aβ42

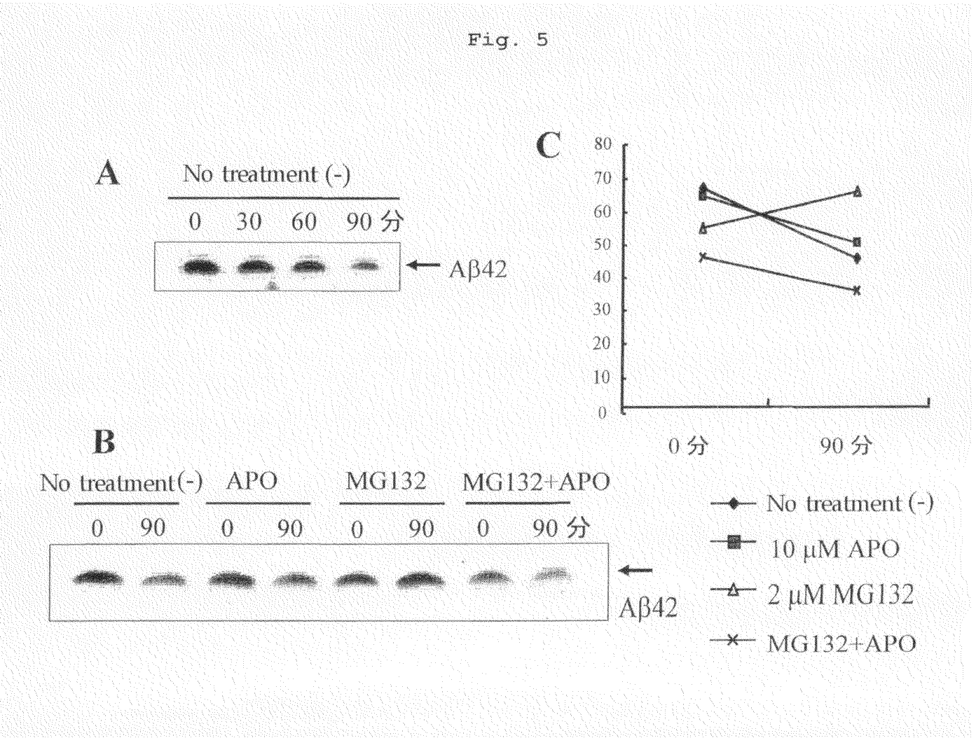

As described in Example 1(1) and (2), degradation of intracellularly accumulated Aβ was assayed for Aβ42 provided that cells were collected 0 minute and 90 minutes after Aβ42 treatment and subjected to immunoblot. As a result, likewise in Aβ40, it was found that degradation after 90 minutes was enhanced by adding APO and that, although degradation was inhibited by adding MG132, the degradation was recovered by adding Aβ42 (FIGS. 5A, 5B, 5C).

### Example 4

### Change in Proteasome Activity by Addition of APO

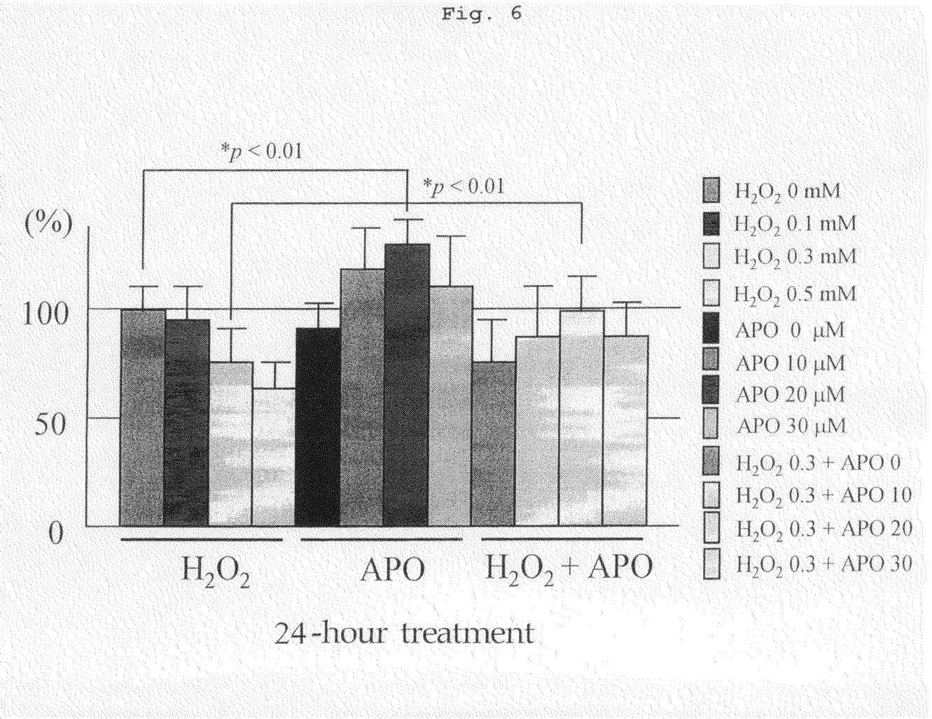

As described in Example 2(1) and (2), a change in the protease activity in SH-SY5Y cells by addition of APO was studied. Measurement of the protease activity was conducted by calculating a relative specific activity by measuring fluorescence generated from degradation of a specific substrate (Suc-LLVY-AMC) using 20S Proteasome Assay Kit (BostonBiochem). Comparison between two groups was done by Student t-test. As a result, it was found that the proteasome activity was increased by APO alone and that the decreased proteasome activity at addition of oxidative stress was recovered by adding APO in SH-SY5Y cells (FIG. 6).

### Example 5

### Protective Effect of APO in Mouse Primary Neuron

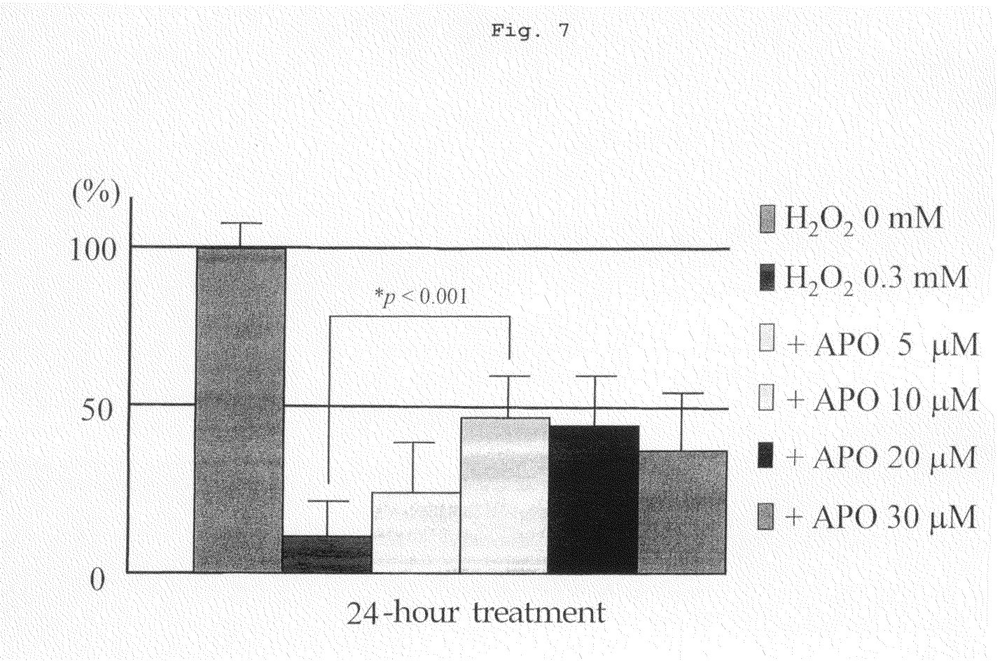

Primary neurons were taken from the brain of fetal mouse of 15 days old, separated by treatment with 0.03% trypsin and cultured in Neurobasal-A+B27 serum-free medium (Gibco). After stabilization by culture for about a week, the reagents were added and a rate of cell survival was measured. As a result, it was found that the apoptosis inhibitory activity of APO observed for oxidative stress in SH-SY5Y cells was also seen in primary neurons from mouse fetus, i.e. native neurons rather than cancerous cells wherein the effect of APO was at the peak at 10 uM and thereafter declined (FIG. 7).

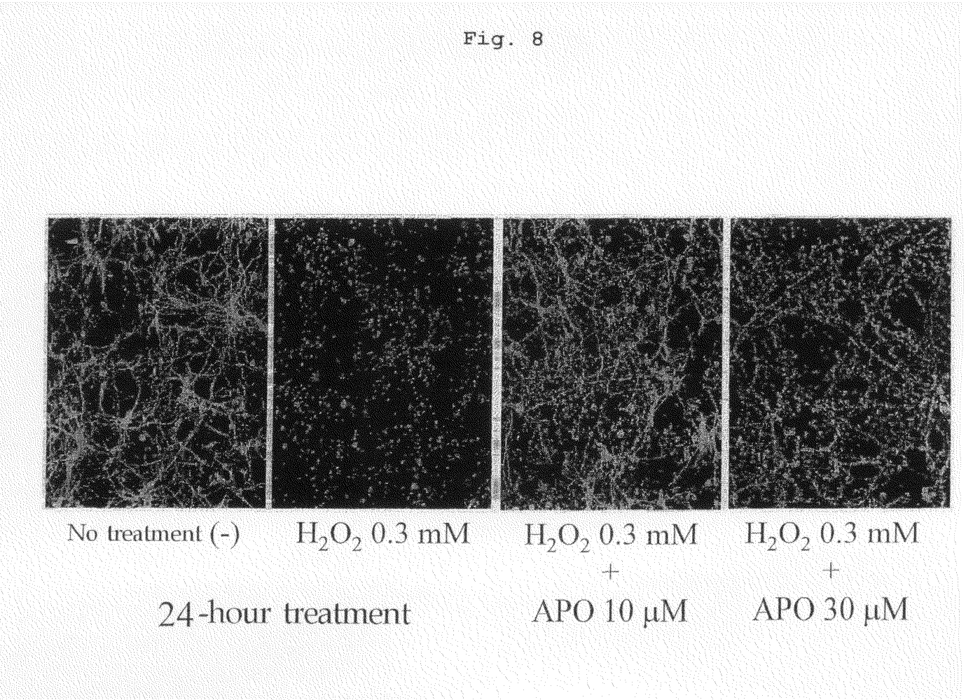

Further, the above mouse primary neurons were immunostained with an antibody to beta-tubulin, a cytoskeletal protein of the axon and a network of the neurite was observed (FIG. 8). As a result, it was found that the addition of APO evidently had the effect not only on cell survival but also protection and maintenance of the network. This effect, viewing that the neuronal network is extensively destroyed in the cerebral cortex in Alzheimer disease patients, strongly suggests efficacy of APO.

### Example 6

### Effect of APO to Ameliorate Recognition Function in 3×Tg Mice

### (1) 3×Tg Mouse

3×Tg mouse is a model mouse of Alzheimer disease (AD) developed by Oddo and LaFerla et al. at University of California, Irvine. It is a mouse having three potent causes of disease wherein a gene of an amyloid precursor protein (APP) with KM670/671NL mutation, which is a gene causative of Swedish familial AD, and a gene of a tau protein (Tau) with P301L mutation, which is one of genes causative of familial frontotemporal dementia (FTD), are incorporated into a knock-in mouse of presenilin-1 (PS1) gene with M146V mutation causative of familial AD. As compared to the conventional model mouse in which a single mutated gene such as e.g. mutated APP is introduced, 3×Tg mouse more readily progresses the disease and exhibits prominent accumulation of amyloid β proteins (Aβ) and phosphorylated tau proteins in neurons, inter alia of the hippocampus and the cerebral cortex, and with the accumulation, shows defects in recognition function, typically memory. It is also reported that the memory function of 3×Tg mouse is recovered when amyloid β proteins accumulated within neurons are decreased but defects in memory relapse if amyloid β proteins become accumulated (Billings LM et al., Neuron 45: 675-688, 2005). Thus, 3×Tg mouse is a model mouse of disease highly suitable for investigating efficacy of a medicament which targets intracellularly accumulated amyloid β proteins.

### (2) Administration of Apomorphine (APO)

For investigating efficacy of apomorphine (APO) hydrochloride administered in vivo, APO was subcutaneously injected to 3×Tg. APO was purchased from Sigma Co. To heterozygous 3×Tg (6 month old; 4 mice in each group) generated from crossbreeding of a normal mouse and a Tg mouse, each 5 mg/kg of saline or APO was subcutaneously injected weekly for 4 weeks. The animals underwent analysis by Morris water maze test to evaluate the capacity of space memory before injection and after completion of four injections.

### (3) Evaluation of Recognition Function

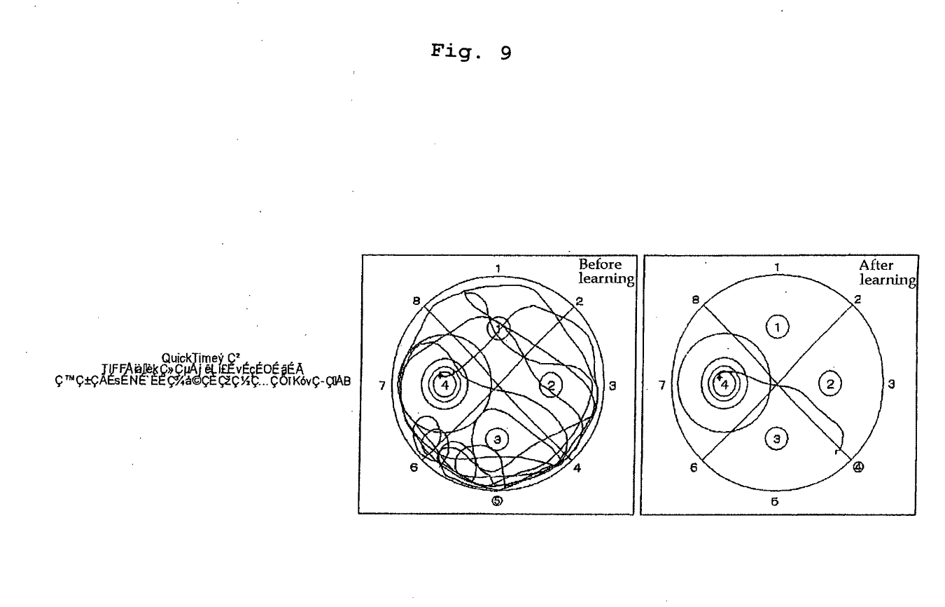

For quantitatively evaluating the capacity of space memory relating to the memory function of the hippocampus, Morris water maze test was performed. An analytical instrument used was DV-Track Video Tracking System (Muromachi Kikai Co., Ltd.). For Morris water maze, a round-shaped pool with a diameter of 1 m is prepared and a transparent goal stand is set in an arbitrary position of the pool. The animals undergo swimming training repeatedly by starting at an arbitrary position for reaching the goal stand (FIG. 9, left panel). In case that the animals could not reach the goal stand within 60 seconds, the test is discontinued and the animals are put on the goal stand for them to memorize the position. This training is repeated four times a day for three consecutive days and as a consequence the animals come to memorize the position of the goal stand (FIG. 9, right panel). Forty eight hours after completion of learning with time for reaching the goal stand being 15 seconds or less, the animals were let to swim in the pool without the goal stand and three indexes for memory were: time lag (s) in reaching the position where the goal stand was originally placed, a frequency (n) of crossing the position as seeking for the goal stand, and residential time (%) within ¼ block of the position where the goal stand was originally placed (48-hour probe test).

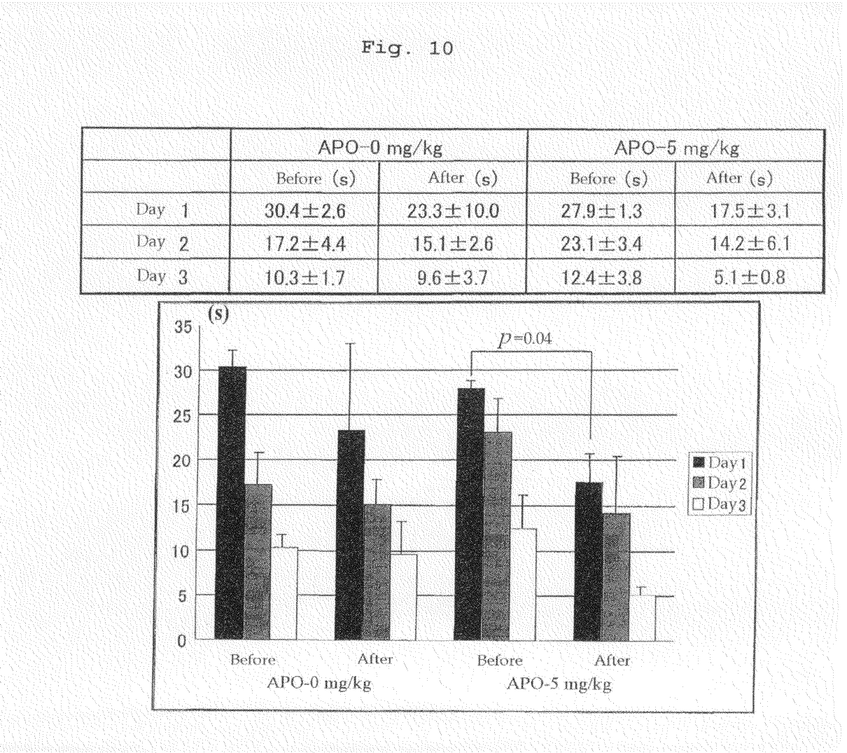

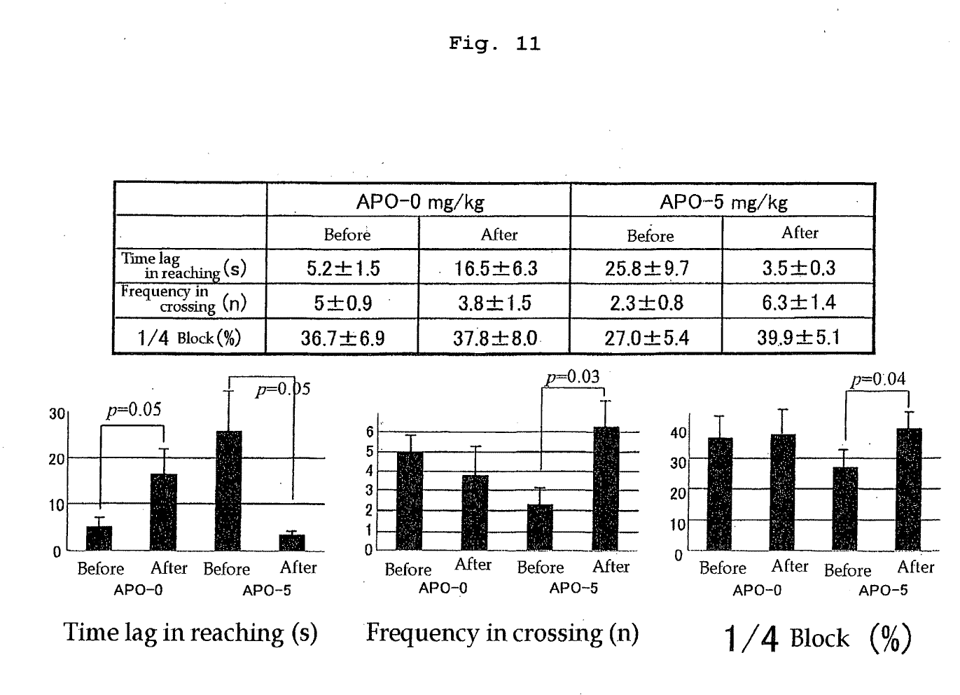

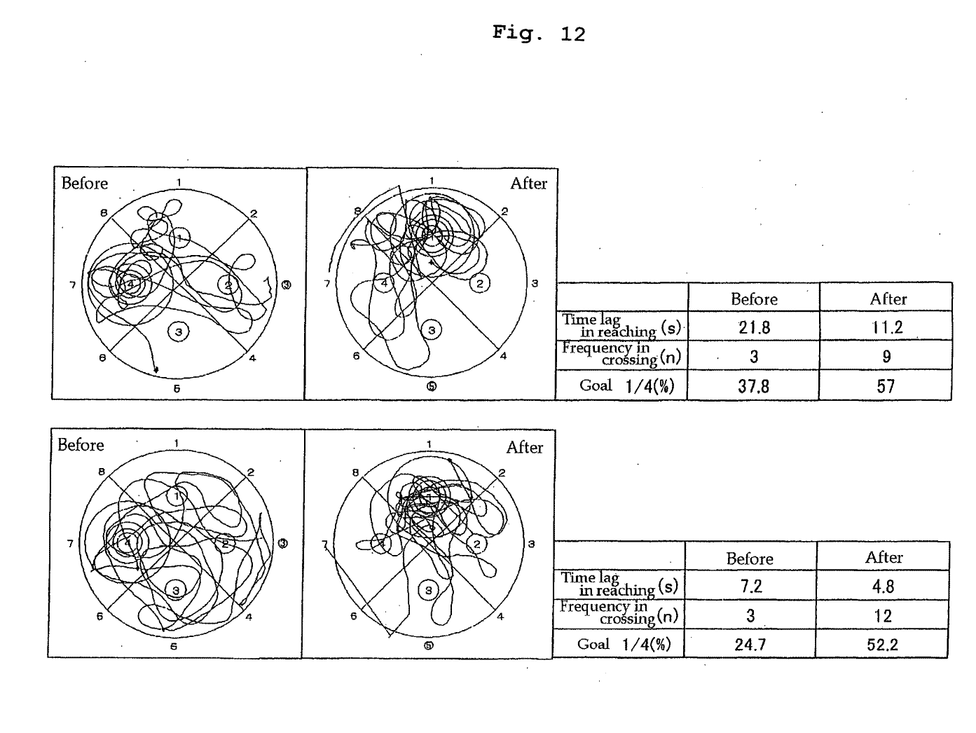

As a result, for the capacity of learning after 3-day consecutive training, both control group and APO injection group exhibited similar capacity of learning but with a tendency that the latter had slightly higher capacity of learning after treatment than the former (FIG. 10). In 48-hour probe test, shortening of time lag in reaching the goal stand (p=0.05), increase in frequency of crossing the goal stand (p=0.03), and increase in residential time (%) within ¼ block of the goal stand (p=0.04) were significantly observed in APO injection group (FIG. 11). In contrast, in control group, time lag in reaching the goal stand was significantly prolonged (p=0.05), frequency of crossing the goal stand tended to decrease and residential time (%) within ¼ block of the goal stand did not change (FIG. 11). Thus, improvement in the capacity of short-term space memory by APO injection was suggested from these indexes. FIG. 12 shows loci of swimming of mice as demonstrating evident alleviation in these indexes after injection of APO. It shows that the capacity of memory for the position where the goal stand was originally placed was markedly improved.

### Example 7

### Pathological Evaluation in 3×Tg Mouse with APO Administration

In 3×Tg mouse, it is observed that defects in the recognition function and intracellularly accumulated amyloid β proteins are correlated to each other. For evaluating amyloid β proteins accumulated within neurons, 3×Tg mice used in Example 6 were sacrificed for pathological analysis. Immunostaining was done with an antibody specific to an amyloid β protein and an antibody specific to a phosphorylated tau protein, which is a major component of neurofibrillary tangle in the AD brain. After completion of Morris water maze test after APO injection, the mice were subjected to perfusion fixation with 4% paraformaldehyde. Then, the whole brain was taken out, fixed with 4% paraformaldehyde and was left to stand in 15% sucrose for 48 hours and in 30% sucrose for 48 hours at 4° C. A slice with 20 μm thickness cut from the forehead including the hippocampus was prepared. Immunostaining was performed with 4G8 (Signet Laboratories, 1:1000) for an anti-amyloid β protein antibody and AT8 (Innogenetics, 1:200) for an anti-phosphorylated tau protein antibody using mouse to mouse immunostaining kit (Zymed) for DAB staining.

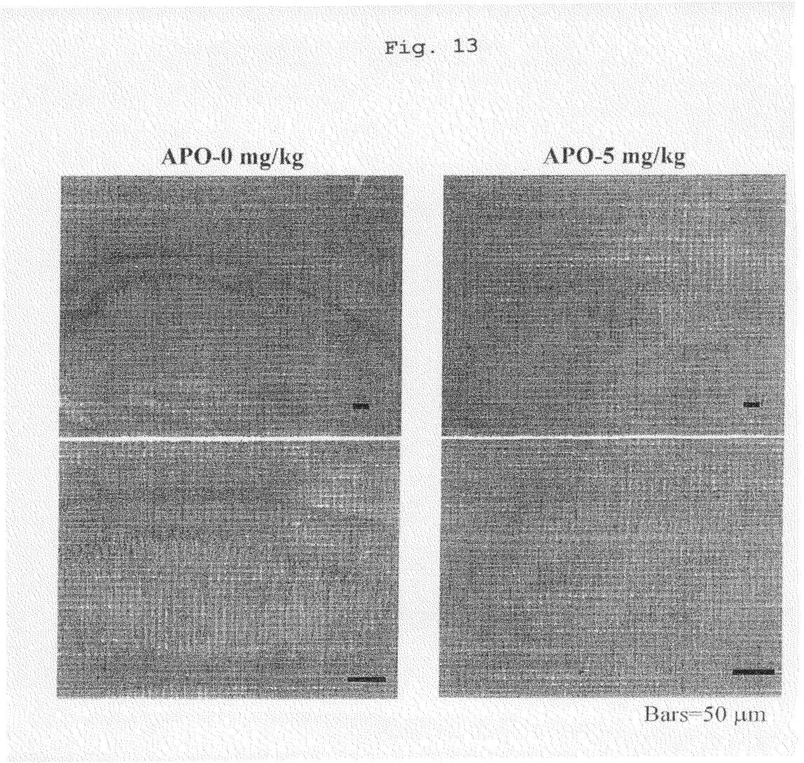

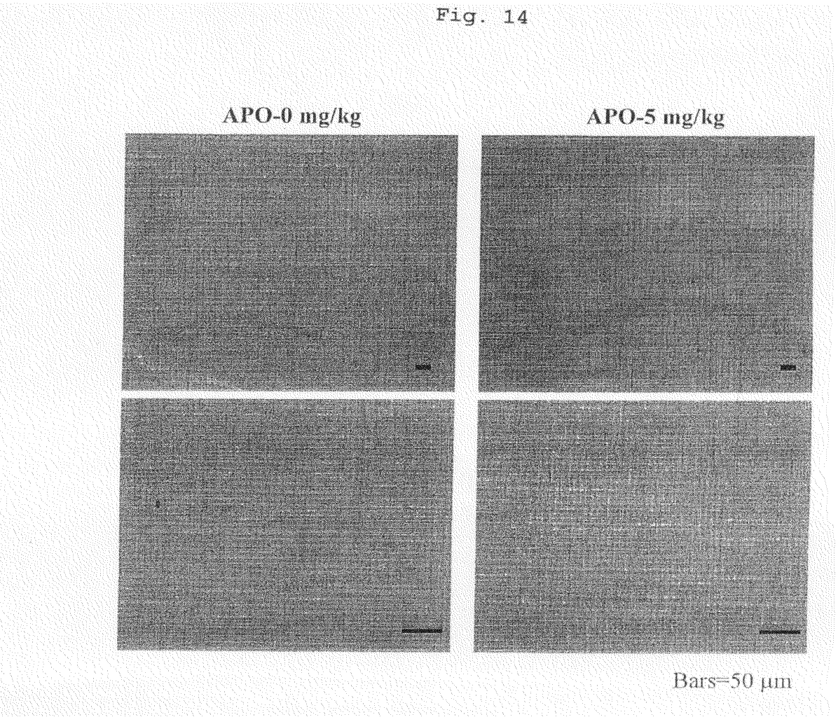

As a result, intraneuronal accumulation of amyloid β proteins was evidently inhibited both in the cerebral cortex and the hippocampus CA1 region (FIG. 13). On the other hand, for 3×Tg mice where accumulation of phosphorylated tau proteins in many neurons of the cerebral cortex and the hippocampus was observed (FIG. 14, left panel), the accumulation was inhibited in mice injected with APO (FIG. 14, right panel).

As a consequence of the results shown above, it was proved that, likewise in in vitro experiment, APO may alleviate the functional defect of neurons by enhancing degradation of amyloid β proteins accumulated within neurons so as to inhibit in vivo the pathological mechanism of AD in 3×Tg.

### Example 8

### Preventive Effect to Onset of AD by AP Administration

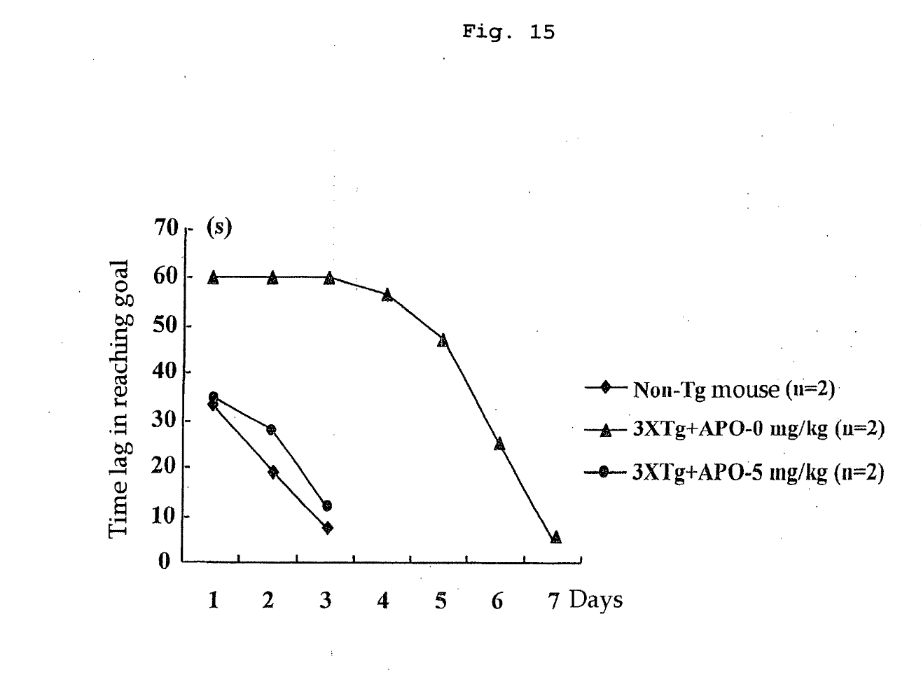

For investigating whether APO administration may prevent onset of AD, the effect of weekly administration of 5 mg/kg of APO before onset was studied. The recognition function and the pathological change in 3×Tg mice may widely vary from individual to individual and thus still much more data would be necessary for obtaining a definite conclusion. However, as shown in FIG. 15, the capacity of memory acquisition in swimming training in homozygous 3×Tg mice which received APO injection at 3 months old up till 6 months old was evidently superior to that of 3×Tg mice with no treatment (APO 0 mg/kg) and was comparable to non-transgenic mice (Non-Tg mice). This result suggests that APO is a potential medicament for prophylaxis of AD.

### INDUSTRIAL APPLICABILITY

The medicament for prophylaxis and treatment of Alzheimer disease comprising as an active ingredient apomorphine hydrochloride according to the present invention may reduce amyloid β proteins and p53 proteins accumulated within neurons, rather than inhibition of coagulation and accumulation of extracellularly accumulated amyloid β proteins as conventionally viewed. Since the intracellular coagulation and accumulation of amyloid β proteins are thought to be the direct cause of massive neuronal death in Alzheimer disease, by enhancing degradation of such amyloid β proteins, the medicament for prophylaxis and treatment of Alzheimer disease of the present invention may exert prominent effects to inhibit neuronal death. Furthermore, as shown in primary neurons, its prominent protective effect to the neuronal network may be construed to be effective in prophylaxis and inhibition of progress of symptoms of dementia in Alzheimer disease. Thus, as compared to a coagulation inhibitor of extracellular amyloid β proteins, the medicament according to the present invention allows for more eradicative prophylaxis and treatment of Alzheimer disease by inhibiting massive neuronal death and destruction of the neuronal network.

Besides, it is known that the amyloid β proteins accumulated within neurons, other than induction of neuronal death via p53 dependent apoptosis, may also be involved in damage to energy production in mitochondria, damage to synaptic function, and damage to proteasome function. In 3×Tg mice, after administration of apomorphine hydrochloride, fall-off of massive neurons was not observed even at the stage of 6-month when amyloid β proteins are accumulated within neurons of the hippocampus and the cerebral cortex. Thus, as a mechanism of improvement in memory power as a consequence of administration of apomorphine hydrochloride, it was suggested that the function of various neurons such as the hippocampus was recovered through decrease in intracellular amyloid β proteins. This also suggests that intracellular accumulation of amyloid β proteins is a preliminary stage of p53 dependent apoptosis and that activation of a chaperon protein called AB-DIP, which transfers amyloid β proteins from cytoplasm to nuclear, would be necessary for inducing massive apoptosis. Accordingly, even prior to occurrence of massive neuronal death, apomorphine hydrochloride may improve the function per se of neurons by reducing intracellularly accumulated amyloid β proteins to thereby contribute amelioration of recognition function of Alzheimer disease patients.

1. A medicament for prophylaxis and treatment of Alzheimer disease comprising as an active ingredient apomorphine hydrochloride.

2. The medicament for prophylaxis and treatment of Alzheimer disease of claim 1 wherein the medicament acts through inhibition of intracellular accumulation of amyloid β and/or p53 dependent cell death.

3. The medicament for prophylaxis and treatment of Alzheimer disease of claim 1 wherein a level of intracellularly accumulated amyloid β proteins and/or phosphorylated tau proteins is lowered to improve energy production, protein metabolism and synaptic function of neurons so as to ameliorate the recognition capacity such as defects of memory.
